(19) United States
(12) Patent Application Publication
(10) Pub. No.: US 2008/0234225 A1
(43) Pub. Date: Sep. 25, 2008

(54) Method of treatment

(76) Inventors: John Lezdey, Indian Rocks Beach, FL (US); Darren Lezdey, Indian Rocks Beach, FL (US)

Correspondence Address: JOHN LEZDEY & ASSOCIATES, 2401 West Bay Drive, Suite 118, Largo, FL, 33770, US

(21) Appl. No.: 10/801,419

(22) Filed: Mar. 15, 2004

(51) Int. Cl. A61K 31/724; A61K 31/573; A61K 31/353; A61K 31/198; A61L 15/16

(52) U.S. Cl. 514/58; 514/456; 514/565; 514/171; 424/444

(57) ABSTRACT

A method and composition for preventing the formation of lesions in a patient having a site of inflammation by the use of a cromolyn compound. The cromolyn compound is used with a composition containing penetrating aids.

### FIELD OF THE INVENTION

A method for inhibiting keratinocyte growth and differentiation and the release of tryptase in skin disease by treatment with cromolyn compound containing compositions. More particularly, there is a method for preventing the formation of skin lesions resulting from presence of tryptase of trypsin.

### BACKGROUND OF THE INVENTION

In skin diseases such as lupus, atopic dermatitis psoriasis, keratosis and the like, skin lesions and scaling form as a result of tissue degradation because of the presence of tryptase, chymase and catepsin-G which are released by the degranulation of mast cells in the acute inflammatory cycle. Other causes of the presences of the mediators of inflammation are the activation of proteinase-activated receptors.

U.S. Pat. No. 6,573,249 to Lezdey et al, which is herein incorporated by reference, discloses the use of cromolyn containing compositions after the lesions have already formed. The compositions are primarily to penetrate through the stratum corneum and treat the lesions.

Although serine proteases are usually considered to act principally as degradative enzymes, certain proteases are signaling molecules that specifically regulate cells by cleaving and triggering members of a new family of proteinase-activated receptors (PARs). There are three members of the family PAR-1, and PAR-3 which are receptors for thrombin, and PAR-2 a receptor for trypsin and mast cell tryptase. Trypsin and mast cell tryptase are involved in inflammation and the cause of lesions in skin diseases, for example atopic dermatitis and keratosis.

The involvement of PAR-2 in inflammation is supported by the finding that PAR-2 mRNA is unregulated by tumor necrosis factor-alpha and interleukin 1-2 which both act in acute inflammatory response.

Mast cell tryptase cleaves and activates many cell types including transfected cells, endothelical cells, enterocytes and colonic myocytes. Tryptase is mitogenic for epithelial cells.

Cromolyn compounds prevent the degranulation of mast cells and control the activation of PAR-2.

It would be desirable to provide a means for preventing or reducing the effect of PAR-2 activation in skin diseases especially in autoimmune diseases such as atopic dermatitis and lupus to prevent the formation of skin lesions.

In atopic dermatitis, lupus and keratosis, there is usually a redness which occurs on the skin which indicates an inflammatory response. Applying a PAR-2 inhibitor at the first signs of inflammation would inhibit epithelial growth and formation of lesions by inhibiting tryptase and chymase activity.

### SUMMARY OF THE INVENTION

The present invention relates to the prevention of the formation of skin lesions by the administration of a cromolyn compound containing composition prior to destruction of tissue leading to the formation of lesions. Advantageously, the composition contains at least 0.5% to 5% by weight of the cromolyn compound. It is further advantageous to provide penetration aids especially when the cromolyn compounds are used in combination with other anti-inflammatory agents.

It is therefore an object of the invention to prevent the occurrence of lesions in inflammatory skin diseases.

It is another object of the invention to prevent the activation of PAR-2.

It is yet another object of the invention to increase the penetration of the cromolyn compounds through the stratum corneum.

It is also an object of the invention to prevent the occurrence of lesions in atopic dermatitis diseases.

### DESCRIPTION OF THE PREFERRED EMBODIMENTS

In diseases and injuries such as lupus, atopic dermatitis, psoriasis, decubitus ulcers and diaper rash, inflammation occurs prior to the formation of lesions or rashes. Degranulation of mast cells results in the release of tryptase, chymase, TNF-alpha, cathepsin-G, histamines, etc. which causes destruction of tissue and proliferation of skin cells. Compositions containing at least 0.5 to 5% by weight of a cromolyn compound can be administered topically at the initiation of an inflammatory response or prior thereto to desensitize PAR-2. The cromolyn compounds prevent the degranulation of mast cells which cause the release of the destructive proteassses or when inflammation has started to shut down the inflammatory cycle.

It is preferable to incorporate the cromolyn compounds in compositions which increase the penetration of the cromolyn compounds into the skin. Increase of penetration can be the result of an occlusive bandage which can be formed by a hydrophilic lipid miscible ointment, including olive oil, mineral oil, and liposome.

Penetration aids include cyclodextrin cationic quaternary ammonium salts, and arginine-containing amino acids, particularly L-arginine.

According to one-embrochment of the present invention, a hydrophilic ointment base is utilized not only to act as an occlusive bandage but to act as a protective barrier. Penetrating agents are used to enhance the penetration through the skin barrier at the first signs of inflammation. Steroids differ in penetration capabilities than cromolyn. Water by itself is a penetrating agent but works differently with each of cromolyn and corticosteroids. Cyclodextrin alpha, beta or gamma or arginine-containing amino acids, particularly L-arginine promote penetration of each of the components. The addition of a cationic quaternary ammonium compound is not only a preservative but also promotes passage through the lipid barrier of the stratum corneum. Other delivering vehicles include olive oil, and liposomes, particularly NOVASOME™ a liposome of Eavsco Corp. of New Jersey.

The cromolyn is generally utilized in an amount of about 0.5 to 5% by weight of composition in the prophylactic treatment of skin diseases. Steroids of up to 0.5% by weight can be used in the cromolyn composition to control leukotriene B-4 and the inflammation.

The hydrophilic lipid miscible base which may be used includes petrolatum, mineral oil, mineral wax, wax wool alcohol and combinations thereof A preferred vehicle contains about 20 to 50% by weight of petrolatum, about 5 to 20% by weight of mineral oil, about 0 to 20% by weight of mineral wax, about 0 to 10% by weight of wool wax alcohol, and about 1 to 10% by weight water.

A preferred composition of the present invention comprises:

1-4% by weight cromolyn

0-0.5% benzalkonium chloride

0-0.5% hydrocortisone acetate

0-2% hyaluronic acid

About 1% cyclodextrin

5-10% water

NOVASOME™ qs

The corticosteroids which may be used include beta-methasone, triamcinolone acetonide, hydrocortisone, prednisone, dexamethasone, fluoroandrenolide, and the like.

The cationic quaternary ammonium salts which include a greater number of short-chain alkyl groups in the structure, incline toward better properties. Specific examples of such compounds that may be used in the compositions of this invention include di-isobutyl cresoxy ethoxy ethyl dimethyl benzyl ammonium chloride, di-isobutyl phenoxy ethoxy ethyl dimethyl benzyl ammonium chloride, myristyl dimethylbenzene ammonium chloride, benzalkonium chloride, cetyl pyridinium chloride, coconut dimethyl benzyl ammonium chloride, stearyl dimethyl benzyl ammonium chloride, alkyl dimethyl benzyl ammonium chloride, alkyl diethyl benzyl ammonium chloride, alkyl dimethyl benzyl ammonium bromide, di-isobutyl phenoxy ethoxy ethyl trimethyl ammonium chloride, di-isobutyl phenoxy ethoxy ethyl dimethyl alkyl ammonium chloride, methyl-dodecyl benzyl trimethyl ammonium chloride, cetyl trimethyl ammonium bromide, octadecyl dimethyl ethyl ammonium bromide, cetyl dimethyl ethyl ammonium bromide, octadecenyl-9-dimethyl ethyl ammonium bromide, dioctyl dimethyl ammonium chloride, dodecylo trimethyl ammonium chloride, octadecyl trimethyl ammonium chloride, octadecyl trimethyl ammonium bromide, hexadecynyl trimethyl ammonium iodine, octyl-trimethyl ammonium fluoride, and mixtures thereof. Other water dispersible salts, such as the acetates, sulfates, nitrates, and phosphates, are effective in place of the halides, but the chlorides and bromides are preferred. Drug delivery from aqueous solutions of cyclodextrins is both diffusion controlled and membrane controlled.

The preferred quaternary ammonium salt for use in the invention is benzalkonium chloride. The amount used is generally about 0.01% to 0.5% by weight of composition. The benzalkonium chloride is commercially available and is sold under the name “BARQUAT or quaternium-15.”

The term “cromolyn” as used herein is meant to include cromolyn sodium, disodium cromolyn and esters thereof

The following examples further illustrate the practice of this invention, but are not intended to be limiting thereof. It will be appreciated that the selection of actual amounts of cromolyn and corticosteroids to be administered to any individual patient (human or animal) will fall within the discretion of the attending physician and will be prescribed in a manner commensurate with the appropriate dosages

will depend on the stage of the disease and like factors uniquely within the purview of the attending physician.

### EXAMPLE 1

To a commercially available ointment containing

| | |
|---|---|
| Cetearyl alcohol | Glycerin |
| | Palm oil glyceride |
| Ceteareth-20 | Mineral oil |
| Petrolatum | Sorbitol |
| Advocate oil | Glyceryl dilaurate |
| Dimetticone | Isopropyl palmitate |
| Stearic acid | Allantoin |
| Squalene | Monoxylnol-9 |
| Sodium Carbomer-941 | Methyl paraben |
| Quaternium-15 | Propyl paraben |
| | Fragrance |

was added an aqueous solution containing 2% cromolyn based on total weight of composition, 0.5% of hydrocortisone acetate based on total weight of composition and 4% percent of water based on total weight of composition. Advantageously, cyclodextrin-beta is included.

The composition can be used by patients with atopic dermatitis at the first signs of inflammation.

### EXAMPLE 2

4 grams of cromolyn was admixed with 0.5 gram of benzalkonium chloride, 0.05 gram of cyclodextrin-alpha and 0.25 gram of hydrocortisone acetate in 10 grams of water. After the cromolyn was completely dissolved, the resulting composition was mixed into 85 grams of NOVASOME™.

The resulting composition can be used to prevent diaper rash or decubitus ulcers.

### EXAMPLE 3

A cosmetic gel is prepared by admixing the following ingredients.

| Ingredients | Wt. % |
|---|---|
| Carbomer 940 | 4.10 |
| Xantham gum | 0.15 |
| Propylene glycol | 51.94 |
| Dipropylene glycol | 10.00 |
| Ethoxydiglycol | 15.00 |
| Dimethylisosorbide | 10.00 |
| Aloe Vera gel | 8.00 |
| Cyclodextrin | 0.05 |
| Cromolyn | 1.76 |
| | 100% |

This composition is useful to reduce to prevent lesions for lupus patients.

### EXAMPLE 4

A cosmetic cream is prepared by mixing the following ingredients:

| Ingredients | Wt. % |
|---|---|
| Glycerol stearate | 8.0 |
| PEG-100 stearate | 2.0 |
| Cetostearyl alcohol | 2.5 |
| Disodium EDTA | 0.1 |
| Benzolkonium chloride | 0.1 |
| Propylene glycol | 6.0 |
| Sorbitan stearate | 0.7 |
| Cromolyn | 2.5 |
| Aloe vera gel | 5.0 |
| Water | 13.5 |
| | 100% |

What is claimed:

1. (canceled)

2. The method of claim 13 wherein said composition comprises at least 0.5 to 5% by weight of said cromolyn compound.

3. The method of claim 1 wherein said cromolyn compound is selected from the group consisting of cromolyn sodium, disodium cromolyn and esters thereof.

4. The method of claim 13 wherein said composition contains stratum corneum penetrating agents.

5. The method of claim 4 wherein said penetrating agents are selected from the group consisting of cationic quaternary ammonium salts, cyclodextrin and an arginine containing amino acids.

6. The method of claim 13 wherein said composition forms an occlusive bandage.

7. (canceled)

8. (canceled)

9. (canceled)

10. (canceled)

11. The method of claim 13 wherein the cromolyn compound is cromolyn sodium.

12. The method of claim 13 wherein said penetrating agent is cyclodextrin.

13. A method for treating a patient suffering from diaper rash or decubitus ulcers which comprises topically administering a therapeutically effective amount of a cromolyn compound in a pharmaceutically acceptable carrier to the site of the diaper rash or decubitus ulcers.
